(19) United States
(12) Patent Application Publication
(10) Pub. No.: US 2019/0309347 A1
(43) Pub. Date: Oct. 10, 2019

(54) METHOD AND KIT FOR DETECTING, DISCRIMINATING AND IDENTIFYING BORRELIA SPECIES PRESENT IN A SAMPLE OF HUMAN OR ANIMAL ORIGIN

(71) Applicants: C.A.L.-LABORATOIRE DE BIOLOGIE VETERINAIRE, Troyes (FR); C.RIS PHARMA, Saint Malo (FR)

(72) Inventors: Pierrick AUVRAY, Saint Malo (FR); Elise DESSAUGE, Saint-Malo (FR); Denis FRITZ, Troyes (FR)

(21) Appl. No.: 16/338,144

(22) PCT Filed: Oct. 2, 2017
(86) PCT No.: PCT/EP2017/074988

§ 371 Date: Mar. 29, 2019

(30) Foreign Application Priority Data
Oct. 3, 2016 (FR) 1659525

(51) Int. Cl. C12Q 1/689

(52) U.S. Cl. CPC C12Q 1/689; C12Q 2600/16

(57) ABSTRACT

The present invention concerns a method for detecting, discriminating and identifying Borrelia species present in a sample, such as a tissue or a fluid, of human or animal origin, and a kit for implementing such a method. The method according to the invention comprises steps implemented by quantitative multiplex PCR including:
(a) a step of detecting the presence of Borrelia in the sample and of distinguishing between the Borrelia in the group causing Lyme disease and the Borrelia in the group causing relapsing fever, then, if a signal is detected in step (a) indicating the presence of Borrelia in the group causing Lyme disease,
(b) a step of detecting and distinguishing, in the sample, the presence of the species B. burgdorferi sensu stricto, B. garinii or B. afzelii and the species B. valaisiana or B. bissettii, or, if a signal is detected in step (a) indicating the presence of Borrelia in the group causing relapsing fever, a step (c) of detecting and distinguishing, in the sample, the presence of the species B. hermsii and B. recurrentis, and the species B. duttonii or B. crocidurae of the group causing fevers, and a step (d) of analyzing the results of the amplifications, the method being further implemented using the sequences SEQ ID NO: 1 to 37.

The present invention relates to a method for detecting, discriminating and identifying Borrelia species present in a sample, such as a tissue or a fluid, of human or animal origin, as well as a kit for implementing such a method.

Borrelia is a genus of bacteria of the spirochete phylum and comprising several species that can be classified into two groups depending on the pathology that they cause: one group causing Lyme disease and one group causing relapsing fever.

Lyme disease is in particular transmitted to humans or animals following a tick bite infected by Borrelia. The bacteria will then be found in the saliva of the tick and may reach the bloodstream. The symptoms of Lyme disease are rash at the bite location, progressing despite any treatment toward symptoms such as flulike condition, neurological symptoms, intense fatigue or irregular heartbeat. The Borrelia responsible for Lyme disease include the following species: B. burgdorferi sensu stricto, B. garinii, B. afzelii, B. spielmanii, B. valaisiana, B. bissettii, B. americana, B. andersonii, B. carolinensis, B. japonica, B. lusitaniae, B. sinica, B. tanukii and B. turdi.

Relapsing fevers are bacterial infections transmitted by louse and tick bites. The fevers can be very intense and cause severe symptoms such as chills, pain, pulmonary complications, and even brain hemorrhages. The Borrelia responsible for relapsing fevers include the following species: B. anserina, B. crocidurae, B. duttonii, B. hermsii, B. hispanica, B. miyamotoi, B. parkeri, B. persica, B. recurrentis, B. turicatae, B. lonestari, B. microti and B. theileri.

Treatment for the pathologies caused by Borrelia is essentially based on taking appropriate antibiotics.

Many tests to detect Borrelia are described in the literature, but many are based on the “restriction fragment length polymorphism” technique, antibody detection using ELISA tests generally confirmed by Western Blot, or DNA target sequence amplification methods. However, the tests available today are not very reliable with a large number of false negatives, only detect certain species, or are not discriminating enough to distinguish closely related bacteria. Furthermore, antibody detection does not make it possible to distinguish between an old contamination or an ongoing contamination, the IgGs being able to remain for several years after recovery.

Lastly, the tests offered today are not very sensitive and are thus a source of false-negative results. Yet the effectiveness of the method for detecting Borrelia and identifying species present in a contaminated sample, as well as the sensitivity of the method, will depend on the speed at which treatment is initiated and the specificity of the treatment as a function of the identified species.

The aim of the present invention is thus to propose a method for detecting Borrelia, in a sample of human or animal origin, that is sensitive, effective, reproducible, specific to Borrelia, and that further makes it possible to discriminate between species of Borrelia, the method being easy to implement.

To that end, the invention relates to a method for detecting the presence of Borrelia in a sample of human or animal origin, discriminating and identifying Borrelia species, by quantitative multiplex PCR, comprising:

(a) a step of detecting the presence of Borrelia in the sample and distinguishing between the Borrelia in the group causing Lyme disease and the Borrelia causing relapsing fever, said step consisting of:
extracting DNA from said sample,
amplifying target nucleotide sequences of said DNA using a pair of primers including the forward primer with sequence SEQ ID NO: 1 and the reverse primer with sequence SEQ ID NO: 2 and two probes consisting of sequences SEQ ID NO: 3 and SEQ ID NO: 4, under conditions allowing amplicon production, and
detecting the presence or absence of Borrelia from the group causing Lyme disease or the group causing relapsing fever by detecting the presence of a fluorescence signal resulting from amplicon formation, then

if a signal is detected in step (a) indicating the presence of Borrelia in the group causing Lyme disease,

(b) a step for detecting and distinguishing, in the sample, the presence of the species B. burgdorferi sensu stricto, B. garinii or B. afzelii and the species B. valaisiana or B. bissettii, said step consisting of:
from the DNA extracted from said sample,
(b1) amplifying target nucleotide sequences of said DNA using primer pairs formed by the forward primer with sequence SEQ ID NO: 8 and the reverse primers with sequences SEQ ID NO: 9 and SEQ ID NO: 10 and three probes consisting of sequences SEQ ID NO: 11, SEQ ID NO: 12 and SEQ ID NO: 13, under conditions allowing amplicon production, and
(b2) detecting the presence or absence of Borrelia belonging to the species B. burgdorferi sensu stricto, B. garinii or B. afzelii by detecting the presence of a fluorescence signal resulting from amplicon formation, and
(b3) amplifying target nucleotide sequences of said DNA using pairs of primers formed by the forward primer with sequence SEQ ID NO: 14, the forward primer with sequence SEQ ID NO: 15 and the reverse primer with sequence SEQ ID NO: 16, and two probes consisting of sequences SEQ ID NO: 17 and SEQ ID NO: 18, under conditions allowing amplicon production, and
(b4) detecting the presence or absence of Borrelia belonging to the species B. valaisiana or B. bissettii by detecting the presence of a fluorescence signal resulting from amplicon formation,

or, if a signal is detected in step (a) indicating the presence of Borrelia in the group causing recurring fever,

(c) a step for detecting and distinguishing, in the sample, the presence of the species B. hermsii and B. recurrentis, and the species B. duttonii or B. crocidurae from the group causing relapsing fever, said step consisting of:
from the DNA extracted from said sample,
(c1) amplifying target nucleotide sequences of said DNA using a pair of primers including the forward primer with sequence SEQ ID NO: 19 and the reverse primer with sequence SEQ ID NO: 20 and a pair of primers including the forward primer with sequence SEQ ID NO: 21 and the reverse primer with sequence SEQ ID NO: 22, and two probes consisting of sequences SEQ ID NO: 23 and SEQ ID NO: 24, under conditions allowing amplicon production, and
(c2) detecting the presence or absence of Borrelia belonging to the species B. hermsii and B. recurrentis, by detecting the presence of a fluorescence signal resulting from amplicon formation, and
(c3) amplifying target nucleotide sequences of said DNA using a pair of primers including the forward primer with sequence SEQ ID NO: 25 and the reverse primer with sequence SEQ ID NO: 26 and a probe consisting of the sequence SEQ ID NO: 27, under conditions allowing amplicon production, and
(c4) detecting the presence or absence of Borrelia belonging to the species B. duttonii or B. crocidurae by detecting the presence of a fluorescence signal resulting from amplicon formation,

(d) analyzing the results of the amplifications,

with the understanding that said method includes the sequences having at least 90% homology with the sequences SEQ ID NO: 1 to 4 and 8 to 27.

The method according to the invention makes it possible to detect the Borrelia present in a sample and classify them either in the group causing relapsing fever (B. anserina, B. crocidurae, B. duttonii, B. hermsii, B. hispanica, B. miyamotoi, B. parkeri, B. persica, B. recurrentis, B. turicatae, B. lonestari, B. microti and B. theileri) or in the group causing Lyme disease (B. burgdorferi sensu stricto, B. garinii, B. afzelii, B. spielmanii, B. valaisiana, B. bissettii, B. americana, B. andersonii, B. carolinensis, B. japonica, B. lusitaniae, B. sinica, B. tanukii and B. turdi) by real-time multiplex quantitative PCR. Furthermore, the method also allows precise identification of the species to which the bacteria present belong. Lastly, the sensitivity of the method is such that it makes it possible to detect one to ten copies in a sample.

To that end, during a first step (a), the gene coding the 16S ribosomal RNA of the bacteria is targeted, using a primer pair shared by all of the species. Probes specific to the group of Borrelia causing Lyme disease and Borrelia causing relapsing fever, respectively, are used in order to classify the Borrelia present in the sample in one of these groups.

If this first step has revealed the presence of Borrelia causing Lyme disease, the following step (b) will make it possible to distinguish and identify Borrelia burgdorferi sensu stricto, garinii and afzelii, as well as valaisiana and bissettii, by targeting the gene coding the flagellin of these species. Primer pairs shared between Borrelia burgdorferi sensu stricto, B. garinii and B. afzelii, and primer pairs shared between the species B. valaisiana and B. bissettii, are used. Specific probes for each species are used in order to identify the one or more detected species.

If, however, the first step has revealed the presence of Borrelia belonging to the group of Borrelia causing relapsing fever, the following step (c) will make it possible to identify Borrelia from the species B. hermsii and B. recurrentis by respectively targeting the genes coding the flagellin and the 16S ribosomal RNA or to identify the species B. duttonii or B. crocidurae by targeting the gene coding the RecA protein of these species. Primer pairs shared between the Borrelia B. hermsii and B. recurrentis, and primer pairs shared between the species B. duttonii or B. crocidurae, are used. Specific probes for each species are used in order to identify the one or more detected species.

The method can of course be implemented using any sequences having at least 90%, preferably at least 95%, more preferably at least 98%, homology with the sequences SEQ ID NO: 1 to 4 and 8 to 27.

Advantageously, step (a) includes amplifying nucleotide sequences coding the actin of the host using a pair of primers including the forward primer with sequence SEQ ID NO: 5 and the reverse primer with sequence SEQ ID NO: 6 and a probe consisting of the sequence SEQ ID NO: 7, or sequences having at least 90% homology with sequences SEQ ID NO: 5 to 7, under conditions allowing amplicon production, then detecting the presence or absence of said nucleotide sequence coding the actin by detecting the presence of a fluorescence signal resulting from the amplicon formation.

The detection of the gene for the host's actin makes it possible to check the quality of the extraction of the nucleic acids.

According to one embodiment of the invention, steps (b1) and (b2) comprise:
a step (b1a) consisting of amplifying target nucleotide sequences of said DNA using a primer pair including the forward primer with sequence SEQ ID NO: 8 and the reverse primers with sequences SEQ ID NO: 9 and SEQ ID NO: 10 and two probes SEQ ID NO: 12 and SEQ ID NO: 13,
(b2a) detecting the presence or absence of Borrelia belonging to the species B. garinii or B. afzelii by detecting the presence of a fluorescence signal resulting from amplicon formation, and
a step (b1b) consisting of amplifying target nucleotide sequences of said DNA using a primer pair including the forward primer with sequence SEQ ID NO: 8 and the reverse primers with sequences SEQ ID NO: 9 and SEQ ID NO: 10 and a probe SEQ ID NO: 11.
(b2b) detecting the presence or absence of Borrelia belonging to the species B. burgdorferi sensu stricto by detecting the presence of a fluorescence signal resulting from amplicon formation.
In this embodiment, the detections of the species B. garinii or B. afzelii, on the one hand, and the species B. burgdorferi sensu stricto, on the other hand, are done separately.

Advantageously according to the invention, step (d) includes the sequencing of the amplicons generated at the end of each of the steps, i.e., in steps (a), (b) and/or (c).

The informational potential of the sequencing of the obtained amplicons can make it possible to identify additional species relative to those targeted by the quantitative multiplex PCRs. In particular, the following species can be identified by sequencing: B. anserina, B. coriceae, B. lonestari, B. spielmanii, B. andersonii, B. japonica, B. lusitaniae, B. sinica and B. turdi.

According to one embodiment of the invention, the amplification conditions used in step (a) and in steps (c1) and (c3) are an initial denaturation at 95° C. for 5 minutes, then the implementation of 45 cycles each including a denaturation at 94° C. for 15 sec, then hybridization and elongation at 58° C. for 45 sec.

Preferably, the amplification conditions used in step (b1a) are an initial denaturation at 95° C. for 5 minutes, then the implementation of 45 cycles each including a denaturation at 94° C. for 15 sec, then hybridization and elongation at 62° C. for 45 sec.

Preferably, the amplification conditions used in step (b1b) are an initial denaturation at 95° C. for 5 minutes, then the implementation of 45 cycles each including a denaturation at 94° C. for 15 sec, then hybridization and elongation at 60° C. for 45 sec.

Also preferably, the amplification conditions used in step (b3) are an initial denaturation at 95° C. for 5 minutes, then the implementation of 45 cycles each including a denaturation at 94° C. for 15 sec, then hybridization and elongation at 64° C. for 45 sec.

According to one embodiment, the probes with sequence SEQ ID NO: 3, SEQ ID NO: 4, SEQ ID NO: 7, SEQ ID NO: 11, SEQ ID NO: 12, SEQ ID NO: 13, SEQ ID NO: 17, SEQ ID NO: 18, SEQ ID NO: 23, SEQ ID NO: 24 and SEQ ID NO: 27 are marked with a quencher molecule on the 3′ end and a fluorophore on the 5′ end, the detection of a fluorescence signal attesting to an amplification occurring upon the separation of the fluorophore and the quencher molecule, the probes used during a same step (a), (b1), (b3), (c1), (c3) are marked by different fluorochromes.

Advantageously, the fluorophores are chosen from among 6-FAM, VIC, Cy5, HEX and the fluorescence inhibitor is BlackBerry Quencher (BBQ) or a MGB (Minor Groove Binder) molecule associated with a nonfluorescent quencher (NFQ, Eclipse).

According to one feature of the invention, the sample is blood, urine, saliva, a body tissue.

The invention also relates to a nucleotide sequence comprising a nucleotide sequence chosen from among: SEQ ID NO: 1, SEQ ID NO: 2, SEQ ID NO: 5 SEQ ID NO: 6, SEQ ID NO: 8, SEQ ID NO: 9, SEQ ID NO: 10, SEQ ID NO: 14, SEQ ID NO: 15, SEQ ID NO: 16, SEQ ID NO: 19, SEQ ID NO: 20, SEQ ID NO: 21, SEQ ID NO: 22, SEQ ID NO: 25, SEQ ID NO: 26, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, or any sequence having a homology of at least 90%, preferably at least 95%, more preferably at least 98%, with one of said sequences, as well as their respective complementary sequence.

The invention also relates to a probe comprising a nucleotide sequence chosen from among: SEQ ID NO: 3, SEQ ID NO: 4, SEQ ID NO: 7, SEQ ID NO: 11, SEQ ID NO: 12, SEQ ID NO: 13, SEQ ID NO: 17, SEQ ID NO: 18, SEQ ID NO: 23, SEQ ID NO: 24 and SEQ ID NO: 27, or any sequence having a homology of at least 90%, preferably at least 95%, more preferably at least 98%, with one of said sequences, as well as their respective complementary sequence.

According to one embodiment, the 5′ ends of the probes are marked by the 6-FAM, the VIC, the CY5 or the HEX.

The invention further relates to a kit for detecting the presence of Borrelia in a human or animal sample and discriminating and identifying Borrelia species, by quantitative multiplex PCR, comprising:

(i) a pair of primers including the forward primer with sequence SEQ ID NO: 1 and the reverse primer with sequence SEQ ID NO: 2 and two probes consisting of sequences SEQ ID NO: 3 and SEQ ID NO: 4, or any sequence having a homology of at least 90%, preferably at least 95%, more preferably at least 98%, with one of said sequences, or any complementary sequence,

(ii) pairs of primers formed by the forward primer with sequence SEQ ID NO: 8 and the reverse primers with sequence SEQ ID NO: 9 and SEQ ID NO: 10, and three probes consisting of sequences SEQ ID NO: 11, SEQ ID NO: 12 and SEQ ID NO: 13, or any sequence having a homology of at least 90%, preferably at least 95%, more preferably at least 98%, with one of said sequences, or any complementary sequence,

(iii) pairs of primers formed by the forward primer with sequence SEQ ID NO: 14, the forward primer with sequence SEQ ID NO: 15 and the reverse primer with sequence SEQ ID NO: 16, and two probes consisting of sequences SEQ ID NO: 17 and SEQ ID NO: 18, or any sequence having a homology of at least 90%, preferably at least 95%, more preferably at least 98%, with one of said sequences, or any complementary sequence,

(iv) a pair of primers including the forward primer with sequence SEQ ID NO: 19 and the reverse primer with sequence SEQ ID NO: 20 and a pair of primers including the forward primer with sequence SEQ ID NO: 21 and the reverse primer with sequence SEQ ID NO: 22, and two probes consisting of sequences SEQ ID NO: 23 and SEQ ID NO: 24, or any sequence having a homology of at least 90%, preferably at least 95%, more preferably at least 98%, with one of said sequences, or any complementary sequence,

(v) a pair of primers including the forward primer with sequence SEQ ID NO: 25 and the reverse primer with sequence SEQ ID NO: 26 and a probe consisting of sequence SEQ ID NO: 27, or any sequence having a homology of at least 90%, preferably at least 95%, more preferably at least 98%, with one of said sequences, or any complementary sequence,

(vi) as well as all of the reagents necessary to perform quantitative multiplex PCR,

the probes being marked with a quencher molecule on the 3′ end and a fluorophore on the 5′ end.

The invention lastly relates to the use of a kit as previously defined for detecting the presence of Borrelia in a human or animal sample and discriminating Borrelia species by quantitative multiplex PCR.

The kit according to the invention is used to detect Borrelia species B. burgdorferi sensu stricto, B. garinii, B. afzelii, B. valaisiana and B. bissettii from the group causing Lyme disease, and species B. hermsii, B. recurrentis, B. duttonii or B. crocidurae from the group causing relapsing fever. It further makes it possible to identify the species B. anserina, B. coriceae, B. lonestari, B. spielmanii, B. andersonii, B. japonica, B. lusitaniae, B. sinica and B. turdi using the sequencing technique.

As non-limiting examples, the sample may come from humans or animals, such as pets, livestock or wild animals.

The invention is illustrated hereinafter using an exemplary embodiment of the invention provided as a non-limiting illustration.

### EXAMPLE

Step (a)

Step (a) makes it possible to detect the presence of Borrelia in a sample and to determine whether they belong to the group causing relapsing fever (B. anserina, B. crocidurae, B. duttonii, B. hermsii, B. hispanica, B. miyamotoi, B. parkeri, B. persica, B. recurrentis, B. turicatae, B. lonestari, B. microti and B. theileri) or to the group causing Lyme disease (B. burgdorferi sensu stricto, B. garinii, B. afzelii, B. spielmanii, B. valaisiana, B. bissettii, B. americana, B. andersonii, B. carolinensis, B. japonica, B. lusitaniae, B. sinica, B. tanukii and B. turdi). Step (a) is carried out by real-time multiplex quantitative PCR. To that end, the gene coding the 16S ribosomal RNA of these bacteria is targeted. The gene for the host's actin is used as internal standard.

Research has been done in order to determine primers allowing specific detection of Borrelia bacteria and that are shared by all of the species cited above. Probes making it possible to distinguish between the group causing relapsing fever (“Fever probe”) and the group causing Lyme disease (“Lyme probe”) have been determined. To that end, 84 sequences of the gene coding the 16S RNA of 25 species of Borrelia were aligned. Ten forward primers and 7 reverse primers were determined, and the best pair in terms of specificity, absence of formation of primer dimers, generated amplicon size was chosen, then validated by Q-PCR. Two specific probes for the two Borrelia subgroups were defined and tested.

Furthermore, sequencing the amplicon for the gene coding the 16S RNA produced in this step (a) can make it possible to identify three Borrelia species not searched in the later steps. These species are B. anserina, B. coriceae and B. lonestari.

Sequences of Primers and Probes Specific to Borrelia in Step (a):

The sequences of the pair of primers and probes specific to the two groups of Borrelia are indicated in Table 1.

TABLE 1

| Target | | Sequence | |
|---|---|---|---|
| gene | No. | name | sequence |
| 16S | SEQ ID | Borrelia | 5′-CGT TGT TCG GGA TTA |
| RNA | NO: 1 | A forward | TTG G-3′ |
| | SEQ ID | Borrelia | 5′-CAG ATT CCA CCC TTA |
| | NO: 2 | A reverse | CAC CAG-3′ |
| | SEQ ID | Lyme | 5′-6-FAM-ATA GAG GAA GTT |
| | NO: 3 | Probe | A-NFQ/MGB-3′ |
| | SEQ ID | Fever | 5′-VIC-CAT GAC TAG AGT |
| | NO: 4 | Probe | CT-NFQ/MGB-3′ |
| actin | SEQ ID | Actin | 5′-ACC CAC ACT GTG CCC |
| | NO: 5 | forward: | ATC TA-3′ |
| | SEQ ID | Actin | 5′-CTT GAT GTC ACG CAC |
| | NO: 6 | reverse: | GAT TT-3′ |
| | SEQ ID | Actin | 5′-Cy5-GGC TAC AGC TTC |
| | NO: 7 | probe: | ACC ACC AC-BBQ-3′ |

Materials and Method:

1—Preparation of the Samples:

The DNA used for the Q-PCR is extracted from the sample (blood, cerebrospinal fluid, urine, synovial fluid, tissue biopsies, etc.) with the QIAamp DNA Mini Kit (Ref 51304, Qiagen, France) according to the protocol indicated in the user manual. The DNA is eluted in water and assayed by spectrometry before use. The quality of the extraction is verified by the D.O.260/D.O.280 ratio.

2—Q-PCR Conditions:

Two positive controls were done, corresponding to the nucleic sequences of the Borrelia amplified by the primers defined here and contained in a plasmid vector PexA. They were produced by gene synthesis by sending sequences to the company Eurofins. A first calibration range with 7 points from 0.25 ng (10⁸ copies) to 0.25 ag (0.1 copies) of these positive controls makes it possible to obtain a quantification by number of copies of the Borrelia present in a sample.

3—The Mix Necessary for the Q-PCR Reaction of Step (a) is Prepared as Indicated in Table 2:

TABLE 2

| | | | Final |
|---|---|---|---|
| | Quantity | | concentration |
| Borrelia A forward, 20 μM | 0.25 | μl | 200 nM |
| Borrelia A reverse, 20 μM | 0.25 | μl | 200 nM |
| Lyme Probe, 20 μM | 0.25 | μl | 200 nM |
| Fever Probe, 20 μM | 0.25 | μl | 200 nM |
| Mix iTAQ supermix (Ref. 1725131, Biorad) | 12.5 | μl | 1X |
| Water (Ref. BE51200, Lonza) | qs 20 | μl | — |

By reaction (range point, samples or negative control), 20 μl of mix as well as 5 μl of sample (50 ng to 200 ng), water (for the negative control) or range point are deposited (final reaction volume of 25 μl).

The amplification conditions are as follows: initial denaturation at 95° C. for 5 minutes, then the implementation of 45 cycles each including a denaturation at 94° C. for 15 sec, then hybridization and elongation at 58° C. for 45 sec. The detection of FAM and VIC fluorochromes is selected.

The mix necessary for the Q-PCR reaction to detect actin is prepared as follows:

TABLE 3

| | | | Final |
|---|---|---|---|
| | Quantity | | concentration |
| Actin forward, 20 μM | 0.125 | μl | 100 nM |
| Actin reverse, 20 μM | 0.125 | μl | 100 nM |
| Actin Probe, 20 μM | 0.125 | μl | 100 nM |
| Mix iTAQ supermix (Ref. 1725131, Biorad) | 12.5 | μl | 1X |
| Water (Ref. BE51200, Lonza) | qs 20 | μl | — |

By reaction (range point, samples or negative control), 20 μl of mix as well as 5 μl of sample (50 ng to 200 ng), water (for the negative control) or range point are deposited (final reaction volume of 25 μl).

The amplification conditions are 95° C., 5 minutes, then 40 cycles each of denaturation at 94° C. for 15 sec, then hybridization and elongation at 58° C., 30 sec.

The detection of the Cy5 fluorochrome is selected.

4—Results

Under these conditions, the detection limit for Borrelia in the group causing Lyme disease and Borrelia in the group causing relapsing Fever is 1 copy (or 2.5 ag).

The amplicons for Borrelia in the group causing Lyme disease and Borrelia in the group causing relapsing fever is 153 base pairs. The sequences of these Q-PCR products are the following (Table 4):

TABLE 4

| | Sequence | |
|---|---|---|
| No. | name | sequence |
| SEQ ID | amplicon | 5′-CGTTGTTCGGGATTATTGGGCGTA |
| NO: 28 | of | AAGGGTGAGTAGGCGGATATATAAGTC |
| | Borrelia | TATGCATAAAATACCACAGCTCAACTG |
| | for the | TGGAACTATGTTGGAAACTATATGTCT |
| | group | AGAGTCTGATAGAGGAAGTTAGAATTT |
| | causing | CTGGTGTAAGGGTGGAATCTG-3′ |
| | Lyme | |
| | disease | |
| SEQ ID | amplicon | 5′-CGTTGTTCGGGATTATTGGGCGTA |
| NO: 29 | of | AAGGGTGAGTAGGCGGATATGCAAGTC |
| | Borrelia | TATGCGTAAAATACCACAGCTCAACTG |
| | for the | TGGAACTAGCTGGAAACTGCATGACTA |
| | group | GAGTCTGATAGGGGAAGTTAGAATTCC |
| | causing | TGGTGTAAGGGTGGAATCTG-3′ |
| | relapsing | |
| | Fever | |

Step (b)

If step (a) made it possible to determine the presence of Borrelia from the group causing Lyme disease, then step (b) is respectively carried out to determine the presence of the species B. burgdorferi sensu stricto, B. garinii or B. afzelii (steps (b1), (b1a), (b1b) or (b2)), and the species B. valaisiana or B. bissettii (steps (b3) and (b4)), belonging to this group, by real-time multiplex quantitative PCR. To that end, the gene coding the flagellin of these bacteria is targeted.

In order to implement steps (b1) or (b1a) and (b1b), research has been done to determine a pair of primers making it possible to detect the species B. burgdorferi sensu stricto, B. garinii and B. afzelii as well as 3 probes allowing the specific recognition of each of these species. These three probes are called “Burdg ss probe,” “Garinii probe” and “Afzelii probe.” This work was initially based on the differences in the sequences of these three species in the gene coding the flagellin, which are described in the article by Picken R. (1992, Journal of Clinical Microbiology, 30:99-114).

To that end, 57 sequences of the gene coding the flagellin of 11 species of Borrelia were aligned. The best sequences of primers and probes in terms of specificity, absence of formation of primer dimers, generated amplicon size were chosen, then validated by Q-PCR. The probes thus determined here do not target the same differences in sequences as those of Picken R. The probes described by Picken R. being very long (from 49 to 52 base pairs), however, the Burdg ss and Afzelii probes described here respectively have 5 and 8 nucleotides in common with those of Picken R. (i.e., the 3′ ends of the Picken R. probes have 5 to 8 bases in common with the 5′ ends of two of our probes). The two probes allowing the recognition of the garinii species have no shared base.

The sequencing of the amplicons of the gene for the flagellin produced in step (b1) or (b1a) and (b1b) can make it possible to recognize 6 species of Borrelia not searched in the following steps. These species are B. spielmanii, B. andersonii, B. japonica, B. lusitaniae, B. sinica and B. turdi.

In parallel, research was done to determine a pair of primers making it possible to detect the species B. valaisiana or B. bissettii as well as two probes (“Valaisiana probe” and “Bissettii probe”) allowing the specific recognition of each of these species in order to carry out steps (b3) and (b4). This work was inspired by the differences in the sequences of these two species in the gene coding the flagellin, which are described in the article by Jauhlac B. (2000, Journal of Clinical Microbiology, 38:1895-1900). To that end, 57 sequences of the gene coding the flagellin of 11 species of Borrelia were aligned. The best sequences of primers and probes in terms of specificity, absence of formation of primer dimers, generated amplicon size were chosen, then validated by Q-PCR. The probe determined here and allowing the recognition of the valaisiana species has 15 bases in common with those of Jauhlac B., which makes 27. The two probes allowing the recognition of the bissetti species have no shared base.

Sequences of Primers and Probes Specific to Borrelia Species of the Group Causing Lyme Disease for Steps (b1) or (b1a) and (b1b):

The sequences of the pair of primers and probes specific to the species B. burgdorferi sensu stricto, B. garinii or B. afzelii are indicated in Table 5:

TABLE 5

| Target | | Sequence | |
|---|---|---|---|
| gene | No. | name | sequence |
| flagellin | SEQ ID | Borrelia | 5′-GGA GCA AAT CAA |
| | NO: 8 | B forward | GAT GAR GC-3′ |
| | SEQ ID | Borrelia | 5′-ACA GGA GAA TTA |
| | NO: 9 | B reverse | ACT CCA CCY TG-3′ |
| | | n1 | |
| | SEQ ID | Borrelia | 5′-AGG AGA ATT AAC |
| | NO: 10 | B reverse | TCC GCC TTG-3′ |
| | | n2 | |
| | SEQ ID | Burgd ss | 5′-Cy5-TCA AGA GGG |
| | NO: 11 | Probe | TGT TCA ACA GGA AGG |
| | | | AGC-BBQ-3′ |
| flagellin | SEQ ID | Afzelii | 5′-VIC-AGG GTG CTC |
| | NO: 12 | Probe | AAG AAG A-NFQ/MGB-3′ |
| | SEQ ID | Garinii | 5′-6-FAM-CAG GCT GCT |
| | NO: 13 | Probe | CAG ACT GCA CCT GTT |
| | | | CAA GAA G-ECLIP-3′ |

The Sequences of the Pair of Primers and Probes Specific to the Species B. valaisiana or B. bissettii for Step (b3) are Indicated in Table 6:

TABLE 6

| Target | | Sequence | |
|---|---|---|---|
| gene | No. | name | sequence |
| flagellin | SEQ ID | Borrelia | 5′-ACC AAG ATG AAG |
| | NO: 14 | C forward | CTA TTG CTG TAA-3′ |
| | | n1 | |
| | SEQ ID | Borrelia | 5′-AAG ATG AGG CGA |
| | NO: 15 | C forward | TTG CTG TAA-3′ |
| | | n2 | |
| | SEQ ID | Borrelia | 5′-CAG GTG CTG GYT |
| | NO: 16 | C reverse | GTT GAG C-3′ |
| | SEQ ID | Valaisiana | 5′-VIC-CTA CAC CTG |
| | NO: 17 | Probe | TTC AAG AA-NFQ/MGB- |
| | | | 3′ |
| flagellin | SEQ ID | Bissettii | 5′-6-FAM-TCG CAA |
| | NO: 18 | Probe | ATC TTT TCT CT- |
| | | | NFQ/MGB-3′ |

Protocol:

1—Preparation of the Samples:

The preparation of samples is identical to that done in step (a).

2—Q-PCR Conditions:

Five positive controls were done. They correspond to the nucleic sequences of the Borrelia amplified by the two pairs of primers defined here and contained in a plasmid vector PexA. A first calibration range with 7 points from 0.25 ng (10⁸ copies) to 0.25 ag (0.1 copies) of these positive controls makes it possible to obtain a quantification by number of copies of the Borrelia present in a sample.

3—Steps (b1a) and (b1b):

Steps (b1a) and (b1b) aim to detect the presence or absence of Borrelia belonging to the species B. burgdorferi sensu stricto, B. garinii or B. afzelii by detecting the presence or absence of a fluorescence signal resulting from amplicon formation.

Step (b1a) aims to amplify target nucleotide sequences using a primer pair including the forward primer with sequence SEQ ID NO: 8 and the reverse primers with sequences SEQ ID NO: 9 and SEQ ID NO: 10 and two probes consisting of sequences SEQ ID NO: 12 and SEQ ID NO: 13.

The mix necessary for the Q-PCR reaction of step (b1a) is prepared as follows:

TABLE 7

| | | | Final |
|---|---|---|---|
| | Quantity | | concentration |
| Borrelia B forward, 20 μM | 0.25 | μl | 200 nM |
| Borrelia B reverse n1, 20 μM | 0.25 | μl | 200 nM |
| Borrelia B reverse n2, 20 μM | 0.25 | μl | 200 nM |
| Afzelii Probe, 20 μM | 0.25 | μl | 200 nM |
| Garinii Probe, 20 μM | 0.25 | μl | 200 nM |
| Mix iTAQ supermix (Ref. 1725131, Biorad) | 12.5 | μl | 1X |
| Water (Ref. BE51200, Lonza) | qs 20 | μl | — |

By reaction (range point, samples or negative control), 20 μl of mix as well as 5 μl of sample, water (for the negative control) or range point are deposited (final reaction volume of 25 μl).

The amplification conditions are as follows: initial denaturation at 95° C. for 5 minutes, then the implementation of 45 cycles each including a denaturation at 94° C. for 15 sec, then hybridization and elongation at 62° C. for 45 sec.

The detection of FAM and VIC fluorochromes is selected in order to carry out step (b1a).

Step (b1b) aims to amplify target nucleotide sequences using a primer pair including the forward primer with sequence SEQ ID NO: 8 and the reverse primers with sequences SEQ ID NO: 9 and SEQ ID NO: 10 and a probe consisting of sequence SEQ ID NO: 11.

The mix necessary for the Q-PCR reaction of step (b1b) is prepared as follows:

TABLE 8

| | | | Final |
|---|---|---|---|
| | Quantity | | concentration |
| Borrelia B forward, 20 μM | 0.25 | μl | 200 nM |
| Borrelia B reverse n1, 20 μM | 0.25 | μl | 200 nM |
| Borrelia B reverse n2, 20 μM | 0.25 | μl | 200 nM |
| Burdorferiss Probe, 20 μM | 0.25 | μl | 50 nM |
| Mix iTAQ supermix (Ref. 1725131, Biorad) | 12.5 | μl | 1X |
| Water (Ref. BE51200, Lonza) | qs 20 | μl | — |

By reaction (range point, samples or negative control), 20 μl of mix as well as 5 μl of sample, water (for the negative control) or range point are deposited (final reaction volume of 25 μl).

The amplification conditions are as follows: initial denaturation at 95° C. for 5 minutes, then the implementation of 45 cycles each including a denaturation at 94° C. for 15 sec, then hybridization and elongation at 60° C. for 45 sec.

The detection of the Cy5 fluorochrome is selected to carry out step (b1b).

In one variant of the invention, steps b1a and b1b are carried out concomitantly in a single step b1.

4—Steps (b3) and (b4)

Step (b3) aims to amplify target nucleotide sequences of said DNA using a pair of primers including the forward primer with sequence SEQ ID NO: 14, the forward primer with sequence SEQ ID NO: 15 and the reverse primer with sequence SEQ ID NO: 16, and two probes consisting of sequences SEQ ID NO: 17 and SEQ ID NO: 18, under conditions allowing amplicon production.

Step (b3) aims to detect the presence or absence of Borrelia belonging to the species B. valaisiana or B. bissettii by detecting the presence or absence of a fluorescence signal resulting from amplicon formation.

The mix necessary for the Q-PCR reaction of step (b3) is prepared as follows:

TABLE 9

| | Quantity | | Final concentration |
|---|---|---|---|
| Borrelia C forward n1, 20 μM | 0.25 | μl | 200 nM |
| Borrelia C forward n2, 20 μM | 0.25 | μl | 200 nM |
| Borrelia C reverse, 20 μM | 0.25 | μl | 200 nM |
| Valaisiana Probe, 20 μM | 0.25 | μl | 200 nM |
| Bissettii Probe, 20 μM | 0.25 | μl | 200 nM |
| Mix iTAQ supermix (Ref. 1725131, | 12.5 | μl | 1X |
| Biorad) | | | |
| Water (Ref. BE51200, Lonza) | qs 20 | μl | — |

By reaction (range point, samples or negative control), 20 μl of mix as well as 5 μl of sample, water (for the negative control) or range point are deposited (final reaction volume of 25 μl).

The amplification conditions are as follows: initial denaturation at 95° C. for 5 minutes, then the implementation of 45 cycles each including a denaturation at 94° C. for 15 sec, then hybridization and elongation at 64° C. for 45 sec.

The detection of FAM and VIC fluorochromes is selected in step (b4).

5—Results

Under these conditions, the detection limit for the species B. burgdorferi sensu stricto, B. garinii and B. afzelii is 1 copy (or 2.5 ag) and that of the species B. valaisiana and B. bissettii is 1 copy (or 2.5 ag).

The amplicons of the obtained Borrelia species burgdorferi sensu stricto, afzelii and garinii are 180 or 182 base pairs. The amplicons of the obtained Borrelia species valaisiana and bissettii are 135 and 138 base pairs. The sequences of these Q-PCR products are the following (Table 10):

TABLE 10

| | Sequence | |
|---|---|---|
| No. | name | sequence |
| SEQ ID | amplicon | 5′-GGAGCAAACCAAGATGAAGCTATT |
| NO: 30 | of the | GCTGTAAATATTTATGCAGCTAATGTT |
| | Borrelia | GCAAATCTTTTCTCTGGTGAGGGAGCT |
| | burgdorferi | CAAACTGCTCAGGCTGCACCGGTTCAA |
| | sensu | GAGGGTGTTCAACAGGAAGGAGCTCAA |
| | stricto | CAGCCAGCACCTGCTACAGCACCTTCT |
| | | CAAGGCGGAGTTAATTCTCCT(GT)-3′ |
| SEQ ID | amplicon | 5′-GGAGCAAATCAAGATGAAGCAATT |
| NO: 31 | of the | GCTGTAAATATTTATTCAGCTAATGTT |
| | Borrelia | GCAAATCTTTTTGCTGGTGAGGGAGCT |
| | afzelii | CAAGCTGCTCAGGCTGCACCTGTTCAA |
| | | GAGGGTGCTCAAGAAGAAGGAGCTCAG |
| | | CAACCAACACCTGCTACAGCACCTACT |
| | | CAAGGTGGAGTTAATTCTCCT(GT)-3′ |
| SEQ ID | amplicon | 5′-GGAGCAAATCAAGATGAAGCGATT |
| NO: 32 | of the | GCTGTAAATATTTATGCTCTAATGTTG |
| | Borrelia | CAAATCTATTCTCTGGTGAAGGAGCTC |
| | garinii | AGGCTGCTCAGACTGCACCTGTTCAAG |
| | | AAGGTGCTCAACAAGAAGGAGCTCAAC |
| | | AACCAGCACCTGCTACAGCGCCTTCTC |
| | | AGGGTGGAGTTAATTCTCCT(GT)-3′ |
| SEQ ID | amplicon | 5′-AAGATGAGGCGATTGCTGTAAATA |
| NO: 33 | of the | TTTATGCAGCTAATGTTGCAAATCTGT |
| | Borrelia | TTTCTGGTGAGGGAGCTCAAACTGCTC |
| | valaisiana | AGGCTACACCTGTTCAAGAAGGTGCTC |
| | | AACAGGAAGGAGCTCAACAACCAGCAC |
| | | CTG-3′ |
| SEQ ID | amplicon | 5′-ACCAAGATGAAGCTATTGCTGTAA |
| NO: 34 | of the | ATATTTATGCAGCTAATGTCGCAAATC |
| | Borrelia | TTTTCTCTGGTGAGGGAGCTCAAACTG |
| | bissettii | CTCAGGCTGCACCTGTTCAAGAAGGTG |
| | | TTCAGCAAGAAGGAGCTCAACAGCCAG |
| | | CACCTG-3′ |

Step (c)

If step (a) made it possible to determine the presence of Borrelia from the group causing relapsing fever, then step (c) is respectively carried out to determine the presence of the species B. hermsii and B. recurrentis (steps (c1) and (c2)), and the species B. duttonii or B. crocidurae (steps (c3) and (c4)), by real-time multiplex quantitative PCR.

B. hermsii is distinguished by targeting the gene coding the flagellin and B. recurrentis by targeting that coding the 16S rRNA. B. duttonii or B. crocidurae are identified by targeting the RecA gene (DNA repair protein).

In order to carry out step (c1), research was done to determine a pair of primers (Borrelia D fla forward and reverse) and a specific probe (“Hermsii probe”) making it possible to detect the species B. hermsii. To that end, 59 sequences of the gene coding the flagellin of 12 species of Borrelia were aligned. Additionally, a pair of primers (Borrelia D 16S forward and reverse) and a specific probe making it possible to detect the species B. recurrentis (“Recurrentis probe”) were determined. To that end, 61 sequences of the gene coding the 16S rRNA of 23 species of Borrelia were aligned. The best sequences of primers and probes in terms of specificity, absence of formation of primer dimers, generated amplicon size were chosen, then validated by Q-PCR.

Based on polymorphisms described in the RecA gene of B. duttonii by Cutler S., research was also done on the RecA gene in order to determine a pair of primers and a specific probe (“Dut/Croc probe”) to detect B. duttonii or B. crocidurae species and the implementation of step (c3). This research is based on the alignment of 62 sequences of the RecA gene from 10 Borrelia species. The best sequences of primers and probes in terms of specificity, absence of formation of primer dimers, generated amplicon size were chosen, then validated by Q-PCR.

Sequences of Primers and Probes Specific to Borrelia Species of the Group Causing Relapsing Fever:

The sequences of pairs of primers and probes specific to the species B. hermsii or B. recurrentis for step (c1) are indicated in Table 11:

TABLE 11

| Target | | Sequence | |
|---|---|---|---|
| gene | No. | name | sequence |
| flagellin | SEQ ID | Borrelia | 5′-AAA TCT TTT TGC |
| | NO: 19 | D fla | AGG TGA AGG-3′ |
| | | forward: | |
| | SEQ ID | Borrelia | 5′-CAT CAA CAG CGG |
| | NO: 20 | D fla | TTG TAA CAT-3′ |
| | | reverse | |
| 16S rRNA | SEQ ID | Borrelia | 5′-GGA AAT GAC AAG |
| | NO: 21 | D 16S | GTG ATG ACG-3′ |
| | | forward | |
| | SEQ ID | Borrelia | 5′-GCA GTT TCT AGC |
| | NO: 22 | D 16S | ATA GCT CCA CA-3′ |
| | | reverse | |
| flagellin | SEQ ID | Hermsii | 5′-6-FAM-AAG AGA |
| | NO: 23 | Probe | TAG GAC AGC A- |
| | | | NFQ/EQ-3′ |
| 16S rRNA | SEQ ID | Recurrentis | 5′-HEX-AGT CCC GGC |
| | NO: 24 | Probe | TAA TT-NFQ/EQ-3′ |

The sequences of the pair of primers and the probe specific to the B. duttonii and B. crocidurae species for step (c3) are indicated in Table 12:

TABLE 12

| Target | | Sequence | |
|---|---|---|---|
| gene | No. | name | sequence |
| RecA | SEQ ID | Borrelia | 5′-GAT GGC GAG ATG GGT |
| | NO: 25 | E forward: | GAT ACT-3′ |
| | SEQ ID | Borrelia | 5′-GCA TTT CCT CCA GTA |
| | NO: 26 | E reverse: | GTA GTC TCT G-3′ |
| | SEQ ID | Dut/Croc | 5′-HEX-AGC CAT ACT TTC |
| | NO: 27 | Probe: | A-NFQ/EQ-3′ |

Protocol:

1—Preparation of the Samples:

The preparation of samples is identical to that done in step (a).

2—Q-PCR Conditions:

Three positive controls were done. They correspond to the nucleic sequences of the Borrelia amplified by the three pairs of primers defined here and contained in a plasmid vector PexA. They were produced by gene synthesis by sending sequences to the company Eurofins. A first calibration range with 7 points from 0.25 ng (10⁸ copies) to 0.25 ag (0.1 copies) of these positive controls makes it possible to obtain a quantification by number of copies of the Borrelia present in a sample.

3—Steps (c1) and (c2):

Step (c1) aims to amplify target nucleotide sequences of said DNA using a pair of primers including the forward primer with sequence SEQ ID NO: 19 and the reverse primer with sequence SEQ ID NO: 20 and a pair of primers including the forward primer with sequence SEQ ID NO: 21 and the reverse primer with sequence SEQ ID NO: 22, and two probes consisting of sequences SEQ ID NO: 23 and SEQ ID NO: 24.

Step (c2) aims to detect the presence or absence of Borrelia belonging to the B. hermsii and B. recurrentis species, by detecting the presence or absence of a fluorescence signal resulting from amplicon formation.

The mix necessary for the Q-PCR reaction of step (c1) is prepared as follows:

TABLE 13

| | | | Final |
|---|---|---|---|
| | Quantity | | concentration |
| Borrelia D fla forward, 20 μM | 0.25 | μl | 200 nM |
| Borrelia D fla reverse, 20 μM | 0.25 | μl | 200 nM |
| Borrelia D 16S forward, 20 μM | 0.25 | μl | 200 nM |
| Borrelia D 16S reverse, 20 μM | 0.25 | μl | 200 nM |
| Hermsii Probe, 20 μM | 0.25 | μl | 200 nM |
| Recurrentis Probe, 20 μM | 0.25 | μl | 200 nM |
| Mix iTAQ supermix (Ref. 1725131, Biorad) | 12.5 | μl | 1X |
| Water (Ref. BE51200, Lonza) | qs 20 | μl | — |

By reaction (range point, samples or negative control), 20 μl of mix as well as 5 μl of sample, water (for the negative control) or range point are deposited (final reaction volume of 25 μl).

The amplification conditions are an initial denaturation at 95° C. for 5 minutes, then the implementation of 45 cycles each including a denaturation at 94° C. for 15 sec, then hybridization and elongation at 58° C. for 45 sec.

The detection of FAM and HEX fluorochromes is selected in step (c2).

4—Steps (c3) and (c4):

Step (c3) aims to amplify target nucleotide sequences using a pair of primers including the forward primer with sequence SEQ ID NO: 25 and the reverse primer with sequence SEQ ID NO: 26 and a probe consisting of the sequence SEQ ID NO: 27, under conditions allowing amplicon production.

Step (c4) aims to detect the presence or absence of Borrelia belonging to the B. crocidurae species, by detecting the presence or absence of a fluorescence signal resulting from amplicon formation.
The mix necessary for the Q-PCR reaction of step (c3) is prepared as follows:

TABLE 14

| | | | Final |
|---|---|---|---|
| | Quantity | | concentration |
| Borrelia E forward, 20 μM | 0.25 | μl | 200 nM |
| Borrelia E reverse, 20 μM | 0.25 | μl | 200 nM |
| Dut/Croc Probe, 20 μM | 0.25 | μl | 200 nM |
| Mix iTAQ supermix (Ref. 1725131, Biorad) | 12.5 | μl | 1X |
| Water (Ref. BE51200, Lonza) | qs 20 | μl | — |

By reaction (range point, samples or negative control), 20 μl of mix as well as 5 μl of sample, water (for the negative control) or range point are deposited (final reaction volume of 25 μl).

The amplification conditions are 95° C. for 5 minutes, then 45 cycles each including a denaturation at 94° C. for 15 sec, then hybridization and elongation at 58° C. for 45 sec.

In step (c4), the detection of VIC or HEX fluorochromes is selected.

5—Results

Under these conditions, the detection limit of the B. hermsii and B. recurrentis species is 10 copies (or 25 ag) and that of the B. duttonii and B. crocidurae species is 10 copies (or 25 ag).

The amplicons of the obtained Borrelia hermsii and recurrentis species are 146 and 184 base pairs, respectively. The amplicons of the obtained Borrelia duttonii and crocidurae species are 176 base pairs.

The sequences of these Q-PCR products are the following:

TABLE 15

| | Sequence | |
|---|---|---|
| No. | name | Sequence |
| SEQ ID | amplicon | 5′-AAATCTTTTTGCAGGTGAAGGC |
| NO: 35 | of the | GCTCAGGCTGCTCCAGTGCAAGAGA |
| | Borrelia | TAGGACAGCAAGAGGAAGGTCAAGC |
| | hermsii | AGCTCCAGCTCCAGCAGCAGCTCCA |
| | | GCTCAAGGTGGAGTTAATTCCCCAA |
| | | TTAATGTTACAACCGCTGTTGATG-3′ |
| SEQ ID | amplicon | 5′-GGAAATGACAAGGTGATGACGT |
| NO: 36 | of the | TAATTTATGAATAAGTCCCGGCTAA |
| | Borrelia | TTACGTGCCAGCAGCCGCGGTAATA |
| | recurrentis | CGTAAGGGGCGAGCGTTGTTCGGGA |
| | | TTATTGGGCGTAAAGGGTGAGTAGG |
| | | CGGATATGCAAGTCTGTGCGTAAAA |
| | | TACCACAGCTCAACTGTGGAGCTAT |
| | | GCTAGAAACTGC-3′ |
| SEQ ID | amplicon | 5′-GATGGCGAGATGGGTGATACTC |
| NO: 37 | of the | AAATTGGACTTCAAGCAAGACTTAT |
| | Borrelia | GAGTAAGGCTTTAAGGAAAATTACA |
| | duttonii | GCCATACTTTCAAAATCTAATACTT |
| | and | GTATTATGTTTATTAATCAAATAAG |
| | crocidurae | AATGAAAATTGGTTTAGTCTTTGGT |
| | | AGTCCAGAGACTACTACTGGAGGAA |
| | | ATGC-3′ |

1) A method for detecting the presence of Borrelia in a human or animal sample, and identifying Borrelia species, by quantitative multiplex PCR, comprising:
(a) a step of detecting the presence of Borrelia in the sample and distinguishing between the Borrelia in the group causing Lyme disease and the Borrelia causing relapsing fever, said step consisting of:
extracting DNA from said sample,
amplifying target nucleotide sequences of said DNA using a pair of primers including the forward primer with sequence SEQ ID NO: 1 and the reverse primer with sequence SEQ ID NO: 2 and two probes consisting of sequences SEQ ID NO: 3 and SEQ ID NO: 4, under conditions allowing amplicon production, and
detecting the presence or absence of Borrelia from the group causing Lyme disease or the group causing relapsing fever by detecting the presence of a fluorescence signal resulting from amplicon formation, then
if a signal is detected in step (a) indicating the presence of Borrelia in the group causing Lyme disease,
(b) a step for detecting and distinguishing, in the sample, the presence of the species B. burgdorferi sensu stricto, B. garinii or B. afzelii and the species B. valaisiana or B. bissettii, said step consisting of:
from the DNA extracted from said sample,
(b1) amplifying target nucleotide sequences of said DNA using primer pairs formed by the forward primer with sequence SEQ ID NO: 8 and the reverse primers with sequences SEQ ID NO: 9 and SEQ ID NO: 10 and three probes consisting of sequences SEQ ID NO: 11, SEQ ID NO: 12 and SEQ ID NO: 13, under conditions allowing amplicon production, and
(b2) detecting the presence or absence of Borrelia belonging to the species B. burgdorferi sensu stricto, B. garinii or B. afzelii by detecting the presence of a fluorescence signal resulting from amplicon formation, and
(b3) amplifying target nucleotide sequences of said DNA using pairs of primers formed by the forward primer with sequence SEQ ID NO: 14, the forward primer with sequence SEQ ID NO: 15 and the reverse primer with sequence SEQ ID NO: 16, and two probes consisting of sequences SEQ ID NO: 17 and SEQ ID NO: 18, under conditions allowing amplicon production, and
(b4) detecting the presence or absence of Borrelia belonging to the species B. valaisiana or B. bissettii by detecting the presence of a fluorescence signal resulting from amplicon formation,
or, if a signal is detected in step (a) indicating the presence of Borrelia in the group causing recurring fever,
(c) a step for detecting and distinguishing, in the sample, the presence of the species B. hermsii and B. recurrentis, and the species B. duttonii or B. crocidurae from the group causing relapsing fever, said step consisting of:
from the DNA extracted from said sample,
(c1) amplifying target nucleotide sequences of said DNA using a pair of primers including the forward primer with sequence SEQ ID NO: 19 and the reverse primer with sequence SEQ ID NO: 20 and a pair of primers including the forward primer with sequence SEQ ID NO: 21 and the reverse primer with sequence SEQ ID NO: 22, and two probes consisting of sequences SEQ ID NO: 23 and SEQ ID NO: 24, under conditions allowing amplicon production, and
(c2) detecting the presence or absence of Borrelia belonging to the species B. hermsii and B. recurrentis, by detecting the presence of a fluorescence signal resulting from amplicon formation, and
(c3) amplifying target nucleotide sequences of said DNA using a pair of primers including the forward primer with sequence SEQ ID NO: 25 and the reverse primer with sequence SEQ ID NO: 26 and a probe consisting of the sequence SEQ ID NO: 27, under conditions allowing amplicon production, and
(c4) detecting the presence or absence of Borrelia belonging to the species B. duttonii or B. crocidurae by detecting the presence of a fluorescence signal resulting from amplicon formation,
(d) analyzing the results of the amplifications,
with the understanding that said method includes the sequences having at least 90% homology with the sequences SEQ ID NO: 1 to 4 and 8 to 27.

2) The method according to claim 1, characterized in that step (a) includes amplifying nucleotide sequences coding the actin of the host using a pair of primers including the forward primer with sequence SEQ ID NO: 5 and the reverse primer with sequence SEQ ID NO: 6 and a probe consisting of the sequence SEQ ID NO: 7, or sequences having at least 90% homology with sequences SEQ ID NO: 5 to 7, under conditions allowing amplicon production, then detecting the presence or absence of said nucleotide sequence coding the actin by detecting the presence of a fluorescence signal resulting from the amplicon formation.

3) The method according to claim 1, characterized in that steps (b1) and (b2) comprise:
a step (b1a) consisting of amplifying target nucleotide sequences of said DNA using a primer pair including the forward primer with sequence SEQ ID NO: 8 and the reverse primers with sequences SEQ ID NO: 9 and SEQ ID NO: 10 and two probes SEQ ID NO: 12 and SEQ ID NO: 13,
(b2a) detecting the presence or absence of Borrelia belonging to the species B. garinii or B. afzelii by detecting the presence of a fluorescence signal resulting from amplicon formation, and
a step (b1b) consisting of amplifying target nucleotide sequences of said DNA using a primer pair including the forward primer with sequence SEQ ID NO: 8 and the reverse primers with sequences SEQ ID NO: 9 and SEQ ID NO: 10 and a probe SEQ ID NO: 11.
(b2b) detecting the presence or absence of Borrelia belonging to the species B. burgdorferi sensu stricto by detecting the presence of a fluorescence signal resulting from amplicon formation.

4) The method according to claim 1, characterized in that step (d) includes sequencing the amplicons generating at the end of steps (a), (b) and/or (c).

5) The method according to one of steps 1 to 4, characterized in that the amplification conditions used in step (a) and in steps (c1) and (c3) are an initial denaturation at 95° C. for 5 minutes, then the implementation of 45 cycles each including a denaturation at 94° C. for 15 sec, then hybridization and elongation at 58° C. for 45 sec.

6) The method according to claim 1, characterized in that the amplification conditions used in step (b1a) are an initial denaturation at 95° C. for 5 minutes, then the implementation of 45 cycles each including a denaturation at 94° C. for 15 sec, then hybridization and elongation at 62° C. for 45 sec.

7) The method according to claim 1, characterized in that the amplification conditions used in step (b1b) are an initial denaturation at 95° C. for 5 minutes, then the implementation of 45 cycles each including a denaturation at 94° C. for 15 sec, then hybridization and elongation at 60° C. for 45 sec.

8) The method according to claim 1, characterized in that the amplification conditions used in step (b3) are an initial denaturation at 95° C. for 5 minutes, then the implementation of 45 cycles each including a denaturation at 94° C. for 15 sec, then hybridization and elongation at 64° C. for 45 sec.

9) The method according to claim 1, characterized in that the probes with sequence SEQ ID NO: 3, SEQ ID NO: 4, SEQ ID NO: 7, SEQ ID NO: 11, SEQ ID NO: 12, SEQ ID NO: 13, SEQ ID NO: 17, SEQ ID NO: 18, SEQ ID NO: 23, SEQ ID NO: 24 and SEQ ID NO: 27 are marked with a quencher molecule on the 3′ end and a fluorophore on the 5′ end, the detection of a fluorescence signal attesting to an amplification occurring upon the separation of the fluorophore and the quencher molecule, the probes used during a same step (a), (b1), (b3), (c1), (c3) are marked by different fluorochromes.

10) The method according to claim 1, characterized in that the fluorophores are chosen from among 6-FAM, VIC, Cy5, HEX and the fluorescence inhibitor can be BlackBerry Quencher or a MGB molecule associated with a nonfluorescent quencher such as NFQ.

11) The method according to claim 1, characterized in that the sample is blood, urine, saliva, a body tissue.

12) A probe comprising a nucleotide sequence chosen from among: SEQ ID NO: 3, SEQ ID NO: 4, SEQ ID NO: 7, SEQ ID NO: 11, SEQ ID NO: 12, SEQ ID NO: 13, SEQ ID NO: 17, SEQ ID NO: 18, SEQ ID NO: 23, SEQ ID NO: 24 and SEQ ID NO: 27, as well as their respective complementary sequence.

13) The probe according to claim 12, characterized in that the 5′ ends of the sequences are marked by the 6-FAM, the VIC, the CY5, the HEX.

14) The kit for detecting the presence of Borrelia in a human or animal sample and discriminating and identifying Borrelia species, by quantitative multiplex PCR, comprising:
(i) a pair of primers including the forward primer with sequence SEQ ID NO: 1 and the reverse primer with sequence SEQ ID NO: 2 and two probes consisting of sequences SEQ ID NO: 3 and SEQ ID NO: 4, or any sequence having a homology of at least 90%, preferably at least 95%, more preferably at least 98%, with one of said sequences, or any complementary sequence,
(ii) pairs of primers formed by the forward primer with sequence SEQ ID NO: 8 and the reverse primers with sequence SEQ ID NO: 9 and SEQ ID NO: 10, and three probes consisting of sequences SEQ ID NO: 11, SEQ ID NO: 12 and SEQ ID NO: 13, or any sequence having a homology of at least 90%, preferably at least 95%, more preferably at least 98%, with one of said sequences, or any complementary sequence,
(iii) pairs of primers formed by the forward primer with sequence SEQ ID NO: 14, the forward primer with sequence SEQ ID NO: 15 and the reverse primer with sequence SEQ ID NO: 16, and two probes consisting of sequences SEQ ID NO: 17 and SEQ ID NO: 18, or any sequence having a homology of at least 90%, preferably at least 95%, more preferably at least 98%, with one of said sequences, or any complementary sequence,
(iv) a pair of primers including the forward primer with sequence SEQ ID NO: 19 and the reverse primer with sequence SEQ ID NO: 20 and a pair of primers including the forward primer with sequence SEQ ID NO: 21 and the reverse primer with sequence SEQ ID NO: 22, and two probes consisting of sequences SEQ ID NO: 23 and SEQ ID NO: 24, or any sequence having a homology of at least 90%, preferably at least 95%, more preferably at least 98%, with one of said sequences, or any complementary sequence,
(v) a pair of primers including the forward primer with sequence SEQ ID NO: 25 and the reverse primer with sequence SEQ ID NO: 26 and a probe consisting of sequence SEQ ID NO: 27, or any sequence having a homology of at least 90%, preferably at least 95%, more preferably at least 98%, with one of said sequences, or any complementary sequence,
(vi) as well as all of the reagents necessary to perform quantitative multiplex PCR, the probes being marked with a quencher molecule on the 3′ end and a fluorophore on the 5′ end.

15) A use of a kit as defined in claim 14 for detecting the presence of Borrelia in a human or animal sample and identifying Borrelia species by quantitative multiplex PCR.


SEQUENCE LISTING (37 sequences)

SEQ ID NO: 1 - DNA, 19 bp, Artificial Sequence
  Borrelia 16S RNA forward primer A
cgttgttcgggattattgg

SEQ ID NO: 2 - DNA, 21 bp, Artificial Sequence
  Borrelia16S RNA reverse primerA
cagattccacccttacaccag

SEQ ID NO: 3 - DNA, 13 bp, Artificial Sequence
  Borrelia lyme 16S RNA probe
atagaggaagtta

SEQ ID NO: 4 - DNA, 14 bp, Artificial Sequence
  Borrelia feaver 16S RNA probe
catgactagagtct

SEQ ID NO: 5 - DNA, 20 bp, Artificial Sequence
  actine forward primer
acccacactgtgcccatcta

SEQ ID NO: 6 - DNA, 20 bp, Artificial Sequence
  Actine reverse primer
cttgatgtcacgcacgattt

SEQ ID NO: 7 - DNA, 20 bp, Artificial Sequence
  Actine probe
ggctacagcttcaccaccac

SEQ ID NO: 8 - DNA, 20 bp, Artificial Sequence
  Borrelia flagelline forward primer B
ggagcaaatcaagatgargc

SEQ ID NO: 9 - DNA, 23 bp, Artificial Sequence
  Borrelia falgelline reverse primer B 1
acaggagaattaactccaccytg

SEQ ID NO: 10 - DNA, 21 bp, Artificial Sequence
  Borrelia falgelline reverse prime rB 2
aggagaattaactccgccttg

SEQ ID NO: 11 - DNA, 27 bp, Artificial Sequence
  B. burgdorferi sensu stricto flagelline probe
tcaagagggtgttcaacaggaaggagc

SEQ ID NO: 12 - DNA, 16 bp, Artificial Sequence
  B.afzelii flagelline probe
agggtgctcaagaaga

SEQ ID NO: 13 - DNA, 31 bp, Artificial Sequence
  B. Garinii flagelline probe
caggctgctcagactgcacctgttcaagaag

SEQ ID NO: 14 - DNA, 24 bp, Artificial Sequence
  Borrelia flagelline forward primer C 1
accaagatgaagctattgctgtaa

SEQ ID NO: 15 - DNA, 21 bp, Artificial Sequence
  Borrelia flagelline forward primer C 2
aagatgaggcgattgctgtaa

SEQ ID NO: 16 - DNA, 19 bp, Artificial Sequence
  Borrellia flagelline reverse primer C
caggtgctggytgttgagc

SEQ ID NO: 17 - DNA, 17 bp, Artificial Sequence
  B. Valaisiana flagelline probe
ctacacctgttcaagaa

SEQ ID NO: 18 - DNA, 17 bp, Artificial Sequence
  B. Bissentii flagelline probe
tcgcaaatcttttctct

SEQ ID NO: 19 - DNA, 21 bp, Artificial Sequence
  Borrelia flagelline forward primer D
aaatctttttgcaggtgaagg

SEQ ID NO: 20 - DNA, 21 bp, Artificial Sequence
  Borellia Falgelline reverse primer D
catcaacagcggttgtaacat

SEQ ID NO: 21 - DNA, 21 bp, Artificial Sequence
  Borrealia 16S RNA forward primer
ggaaatgacaaggtgatgacg

SEQ ID NO: 22 - DNA, 23 bp, Artificial Sequence
  Borrelia 16S RNA reverse primer
gcagtttctagcatagctccaca

SEQ ID NO: 23 - DNA, 16 bp, Artificial Sequence
  B. Hermsii Flagelline probe
aagagataggacagca

SEQ ID NO: 24 - DNA, 14 bp, Artificial Sequence
  B. recurrentis 16S RNA probe
agtcccggctaatt

SEQ ID NO: 25 - DNA, 21 bp, Artificial Sequence
  Borrelia Rec A forward primer E
gatggcgagatgggtgatact

SEQ ID NO: 26 - DNA, 25 bp, Artificial Sequence
  Borrelia Rec A reverse primer E
gcatttcctccagtagtagtctctg

SEQ ID NO: 27 - DNA, 13 bp, Artificial Sequence
  B. duttonii et B. crocidurae RecA probe
agccatactttca

SEQ ID NO: 28 - DNA, 153 bp, Borrelia
  Borrelia Lyme
cgttgttcgggattattgggcgtaaagggtgagtaggcggatatataagtctatgcataaaataccacagctcaactgtggaactatgttggaaactatatgtctagagtctgatagaggaagttagaatttctggtgtaagggtggaatctg

SEQ ID NO: 29 - DNA, 152 bp, Borrelia
  Borrelia Feaver
cgttgttcgggattattgggcgtaaagggtgagtaggcggatatgcaagtctatgcgtaaaataccacagctcaactgtggaactagctggaaactgcatgactagagtctgataggggaagttagaattcctggtgtaagggtggaatctg

SEQ ID NO: 30 - DNA, 182 bp, Borrelia burgdorferi
ggagcaaaccaagatgaagctattgctgtaaatatttatgcagctaatgttgcaaatcttttctctggtgagggagctcaaactgctcaggctgcaccggttcaagagggtgttcaacaggaaggagctcaacagccagcacctgctacagcaccttctcaaggcggagttaattctcctgt

SEQ ID NO: 31 - DNA, 180 bp, Borrelia afzelii
ggagcaaatcaagatgaagcaattgctgtaaatatttattcagctaatgttgcaaatctttttgctggtgagggagctcaagctgctcaggctgcacctgttcaagagggtgctcaagaagaaggagctcagcaaccaacacctgctacagcacctactcaaggtggagttaattctcct

SEQ ID NO: 32 - DNA, 179 bp, Borrelia garinii
ggagcaaatcaagatgaagcgattgctgtaaatatttatgctctaatgttgcaaatctattctctggtgaaggagctcaggctgctcagactgcacctgttcaagaaggtgctcaacaagaaggagctcaacaaccagcacctgctacagcgccttctcagggtggagttaattctcct

SEQ ID NO: 33 - DNA, 134 bp, Borrelia valaisiana
aagatgaggcgattgctgtaaatatttatgcagctaatgttgcaaatctgttttctggtgagggagctcaaactgctcaggctaacctgttcaagaaggtgctcaacaggaaggagctcaacaaccagcacctg

SEQ ID NO: 34 - DNA, 138 bp, Borrelia bissettii
accaagatgaagctattgctgtaaatatttatgcagctaatgtcgcaaatcttttctctggtgagggagctcaaactgctcaggctgcacctgttcaagaaggtgttcagcaagaaggagctcaacagccagcacctg

SEQ ID NO: 35 - DNA, 146 bp, Borrelia hermsii
aaatctttttgcaggtgaaggcgctcaggctgctccagtgcaagagataggacagcaagaggaaggtcaagcagctccagctccagcagcagctccagctcaaggtggagttaattccccaattaatgttacaaccgctgttgatg

SEQ ID NO: 36 - DNA, 184 bp, Borrelia recurrentis
ggaaatgacaaggtgatgacgttaatttatgaataagtcccggctaattacgtgccagcagccgcggtaatacgtaaggggcgagcgttgttcgggattattgggcgtaaagggtgagtaggcggatatgcaagtctgtgcgtaaaataccacagctcaactgtggagctatgctagaaactgc

SEQ ID NO: 37 - DNA, 176 bp, Borrelia crocidurae
  Borrelia crocidurae et duttonii
gatggcgagatgggtgatactcaaattggacttcaagcaagacttatgagtaaggctttaaggaaaattacagccatactttcaaaatctaatacttgtattatgtttattaatcaaataagaatgaaaattggtttagtctttggtagtccagagactactactggaggaaatgc